(19) United States
(12) Patent Application Publication
(10) Pub. No.: US 2017/0129874 A1
(43) Pub. Date: May 11, 2017

(54) NOVEL IMPROVED PROCESS FOR PREPARING A TRIAZOLE ANTIFUNGAL AGENT

(71) Applicant: VIRUPAKSHA ORGANICS LIMITED, Hyderabad (IN)

(72) Inventors: Chandramouliswar Reddy GANGAVARAM, Hyderabad (IN); Bala Subba Reddy MAMILLA, Hyderabad (IN); Venkateswar Reddy CHAPPETA, Hyderabad (IN); Narsi Reddy MEDA, Hyderabad (IN); Sivakumar SANGARAPPAN, Hyderabad (IN)

(21) Appl. No.: 15/347,869

(22) Filed: Nov. 10, 2016

(30) Foreign Application Priority Data
Nov. 10, 2015 (IN) 5438/CHE/2015
Nov. 10, 2015 (IN) 5439/CHE/2015

(51) Int. Cl. C07D 401/06

(52) U.S. Cl. CPC C07D 401/06

(57) ABSTRACT

An improved process for the preparation of Efinaconazole of Formula (I) or its salts may include reacting triazole compound of Formula (III) or its salts with compound of Formula (IV) or its salts wherein X represents CH2, O or S, in the presence of an alkali metal halide or alkaline earth metal halides to give compound of Formula (II) or its salts wherein X is as defined above and optionally converting compound of Formula (II) or its salts to Efinaconazole or its salts when X represent O or S.

### FIELD OF THE INVENTION

The present invention relates to novel and improved process for preparing a Triazole Antifungal agent or its pharmaceutically acceptable salts.

The present invention more particularly relates to novel and improved process for preparing Efinaconazole or its pharmaceutically acceptable salts.

### BACKGROUND OF THE INVENTION

Efinaconazole is an azole antifungal indicated for the topical treatment of onychomycosis of the toenails due to Trichophyton rubrum and Trichophyton mentagrophytes. Efinaconazole inhibits fungal lanosterol 14α-demethylase involved in the biosynthesis of ergosterol, a constituent of fungal cell membranes.

Efinaconazole is a topical solution, 10% is a clear colorless to pale yellow solution for topical use which is sold under the brand name JUBLIA®. The chemical name of Efinaconazole is ((2R,3R)-2-(2,4-difluorophenyl)-3-(4-methylenepiperidin-1-yl)-1-(1H-1,2,4-triazol-1-yl) butan-2-ol) and the molecular Formula is C₁₈H₂₂F₂N₄O with a molecular weight of 348.39. The structural Formula of Efinaconazole is:

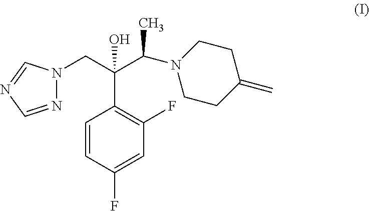 [SMILES: C=C1CCN([C@H](C)[C@](O)(Cn2cncn2)c2ccc(F)cc2F)CC1]

Efinaconazole is disclosed for the first time in U.S. Pat. No. 5,620,994 A. This patent also discloses a process for preparing Efinaconazole, which involves reaction of 4-methylenepiperidine or its HCl salt with 1-(((2R,3S)-2-(2,4-difluorophenyl)-3-methyloxiran-2-yl)methyl)-1H-1,2,4-triazole. The process is shown in the scheme given below:

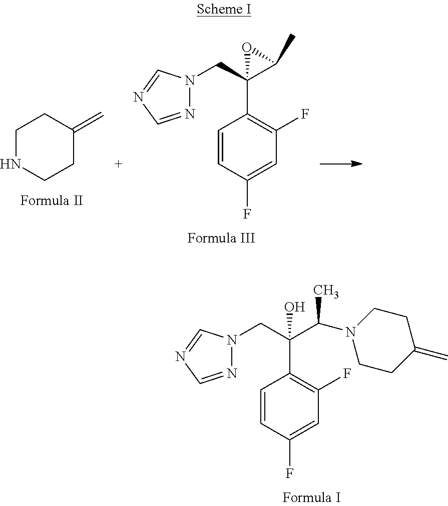

U.S. Pat. No. 8,871,942 B2 discloses a similar process wherein the reaction is carried out in the presence of a hydroxide of an alkali metal or an alkaline earth metal.

In view of the importance acquired by Efinaconazole, there is a great need for developing an alternative, relatively simple, economical and commercially feasible process for the synthesis of Efinaconazole with a commercially acceptable yield and high purity.

It is therefore an object of the present invention to provide a simple, economical and commercially feasible process for the synthesis of Efinaconazole with a commercially acceptable yield and high purity.

### Objective of the Invention

The main objective of the present invention is to provide novel and improved process for the preparation of Efinaconazole or its salts.

Another objective of the present invention is to provide novel intermediates of Efinaconazole or its salts.

Another objective of the present invention is to provide novel and improved process for the preparation of Efinaconazole or its salts which is commercially feasible.

### SUMMARY OF THE INVENTION

Accordingly, the present invention provides an improved process for the preparation of Efinaconazole of Formula (I)

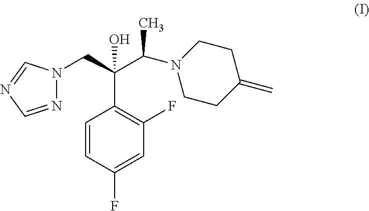 [SMILES: C=C1CCN([C@H](C)[C@](O)(Cn2cncn2)c2ccc(F)cc2F)CC1]

or its salts, which comprises the following steps:
a) reacting triazole compound of Formula (III) or its salts

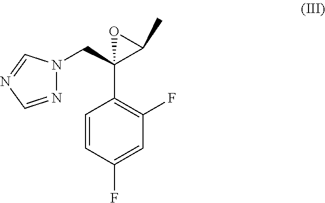 [SMILES: C[C@@H]1O[C@@]1(Cn1cncn1)c1ccc(F)cc1F]

with compound of Formula (IV) or its salts

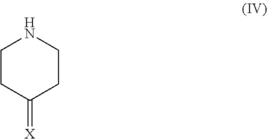

wherein X represents CH₂, O or S, in the presence of an alkali metal halide or alkaline earth metal halides to give compound of Formula (II) or its salts

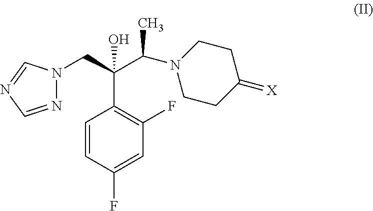

wherein X is as defined above,
b) optionally converting compound of Formula (II) or its salts to Efinaconazole or its salts when X represent O or S.

In a preferred aspect, the present invention provides an improved process for the preparation of Efinaconazole of Formula (I)

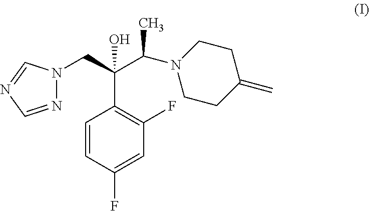 [SMILES: C=C1CCN([C@H](C)[C@](O)(Cn2cncn2)c2ccc(F)cc2F)CC1]

or its salts, which comprises reacting triazole compound of Formula (III) or its salts

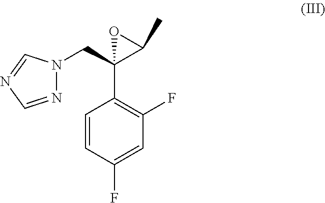 [SMILES: C[C@@H]1O[C@@]1(Cn1cncn1)c1ccc(F)cc1F]

with compound of Formula (IVA) or its salts

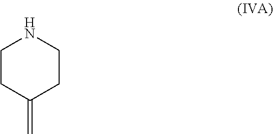

in the presence of an alkali metal halide or alkaline earth metal halides to give Efinaconazole or its salts.

In another preferred aspect, the present invention provides novel process for the preparation of Efinaconazole of Formula (I)

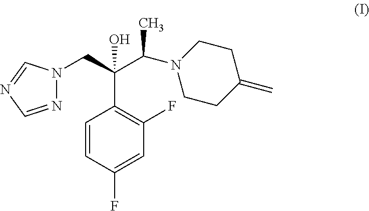 [SMILES: C=C1CCN([C@H](C)[C@](O)(Cn2cncn2)c2ccc(F)cc2F)CC1]

or its salts, which comprises converting compound of Formula (IIA) or its salts

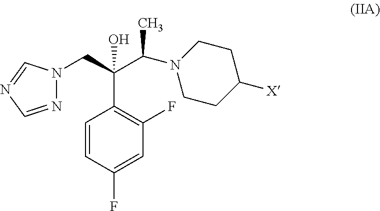

wherein X′ represents O or S, to Efinaconazole or its salts.

In another preferred aspect, the present invention provides novel process for the preparation of Efinaconazole

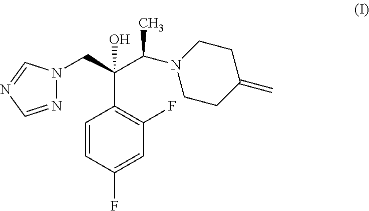 [SMILES: C=C1CCN([C@H](C)[C@](O)(Cn2cncn2)c2ccc(F)cc2F)CC1]

or its salts, which comprises the following steps:
a) reacting triazole compound of Formula (III) or its salts

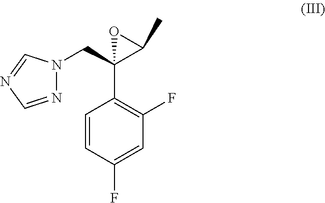 [SMILES: C[C@@H]1O[C@@]1(Cn1cncn1)c1ccc(F)cc1F]

with compound of Formula (IVB) or its salts

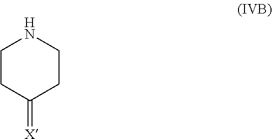

wherein X′ is selected from O or S, to give compound of Formula (IIA) or its salts

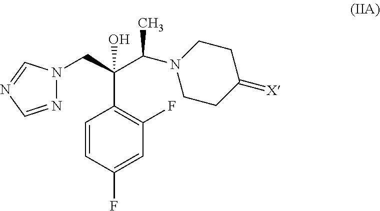

wherein X′ is as defined above,
b) converting compound of Formula (IIA) or its salts to Efinaconazole or its salts.

In yet another preferred aspect, the present invention provides an improved process for the preparation of Efinaconazole

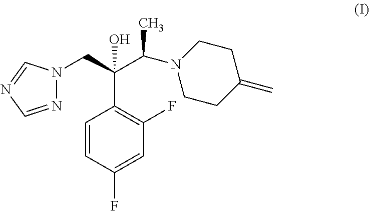 [SMILES: C=C1CCN([C@H](C)[C@](O)(Cn2cncn2)c2ccc(F)cc2F)CC1]

or its salts, which comprises the following steps:
a) converting compound of Formula (V) or its salts

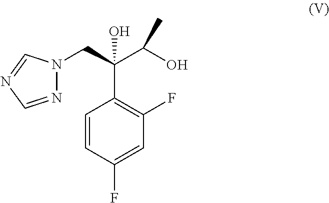 [SMILES: C[C@@H](O)[C@](O)(Cn1cncn1)c1ccc(F)cc1F]

to compound of Formula (VI) or its salts

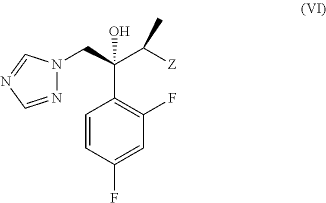

wherein Z is a suitable leaving group,
b) reacting compound of Formula (VI) or its salts with compound of Formula (IV) or its salts

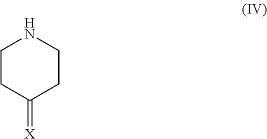

wherein X represents CH₂, O or S, in the presence of an alkali metal halide or alkaline earth metal halides to give compound of Formula (II) or its salts

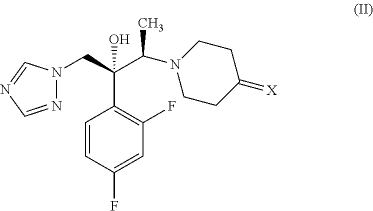

wherein X is as defined above,
c) optionally converting compound of Formula (II) or its salts to Efinaconazole or its salts, when X represents O or S.

In yet another preferred aspect, the present invention provides novel process for the preparation of Efinaconazole

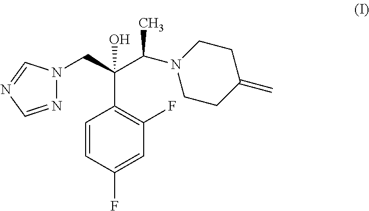 [SMILES: C=C1CCN([C@H](C)[C@](O)(Cn2cncn2)c2ccc(F)cc2F)CC1]

or its salts, which comprises the following steps:
a) converting compound of Formula (V) or its salts

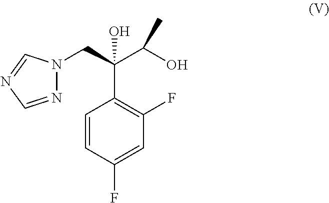 [SMILES: C[C@@H](O)[C@](O)(Cn1cncn1)c1ccc(F)cc1F]

to compound of Formula (VI) or its salts

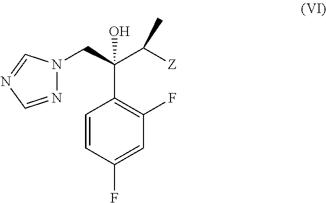

wherein Z is a suitable leaving group,
b) reacting compound of Formula (VI) or its salts with compound of Formula (IVB) or its salts

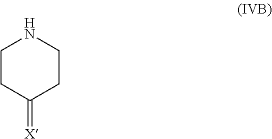

wherein X′ represents O or S, to give compound of Formula (IIA) or its salts

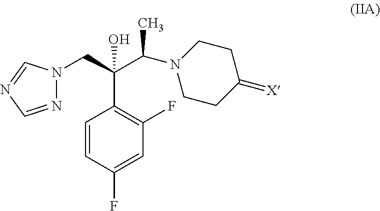

wherein X′ is as defined above,
c) converting compound of Formula (IIA) or its salts to Efinaconazole or its salts.

In yet another preferred aspect, the present invention provides process for the preparation of novel compound of Formula (IIA)

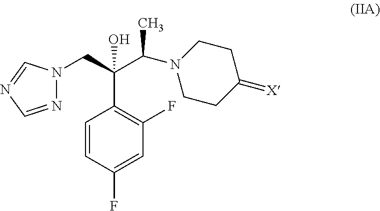

or its salts, wherein X′ represents O or S, which comprises reacting triazole compound of Formula (III) or its salts

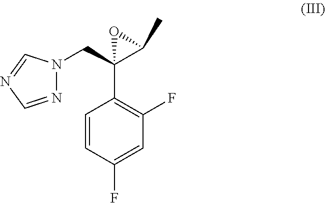 [SMILES: C[C@@H]1O[C@@]1(Cn1cncn1)c1ccc(F)cc1F]

with compound of Formula (IVB) or its salts

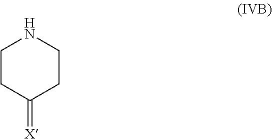

wherein X′ is as defined above, to give compound of Formula (IIA) or its salts.

In yet another preferred aspect, the present invention provides process for the preparation of novel compound of Formula (IIA)

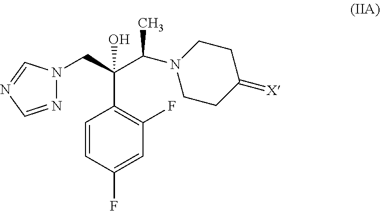

or its salts, wherein X′ represents O or S, which comprises reacting triazole compound of Formula (VI) or its salts

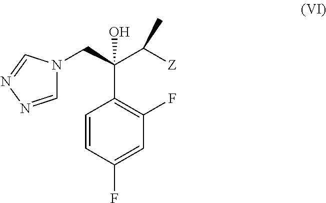

wherein Z is a suitable leaving group, with compound of Formula (IVB) or its salts

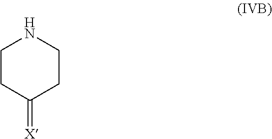

wherein X′ is as defined above, to give compound of Formula (IIA) or its salts.

In yet another aspect, the present invention provides novel process for the preparation of compound of Formula (IIA)

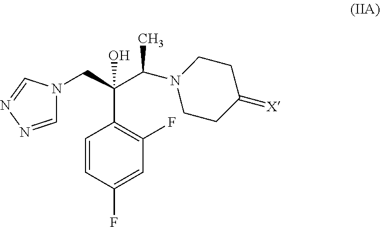

or its salts, wherein X′ represents O or S, which comprises the following steps:
a) converting compound of Formula (V) or its salts

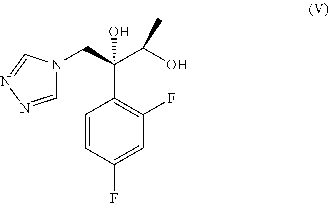 [SMILES: C[C@@H](O)[C@](O)(Cn1cnnc1)c1ccc(F)cc1F]

to compound of Formula (VI) or its salts

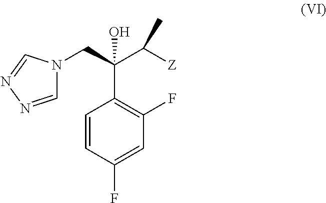

wherein Z is a suitable leaving group,
b) reacting compound of Formula (VI) or its salts with compound of Formula (IVB) or its salts

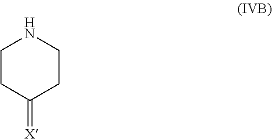

wherein X′ represents O or S, to give compound of Formula (IIA) or its salts

In yet another preferred aspect, the present invention provides novel compound of Formula (IIA) or its salts.

In yet another aspect, the present invention provides the use of compound of Formula (IIA) or its salts in the preparation of Efinaconazole.

### DETAILED DESCRIPTION OF THE INVENTION

Accordingly, the present invention provides novel and improved process for preparing Efinaconazole or its salts.

In a preferred embodiment, the group X in compound of Formula (IV) represents CH₂ which reacts with compound of Formula (III) in the presence of alkali metal halide or alkaline earth metal halides in a solvent to give Efinaconazole or its salts.

In another preferred embodiment, the group X in compound of Formula (IV) represents S or O which reacts with compound of Formula (III) in the presence of a base or in the presence of alkali metal halide or alkaline earth metal halides in a solvent to give compound of Formula (IIA), which is converted to Efinaconazole.

In another preferred embodiment, compound of Formula (IIA) is converted to Efinaconazole or its salts using alkyl triphenylphosphonium salts in a solvent.

Accordingly the present invention provides novel and improved process for preparing Efinaconazole or its salts. The compounds of Formulae II, IIA, III, IV, IVA, IVB, V or V used in the present invention may be isolated or not. Any of the above reactions may be carried out in-situ reactions to obtain Efinaconazole or its salts. The above compounds may be isolated as salts or free bases, if the above compounds are isolated as salts they are converted to their free bases first and used for further reactions.

The crude Efinaconazole as prepared according to the present invention is recrystallized using a suitable solvent to give highly pure Efinaconazole.

Alkali metal halide as used in the present invention is selected from halides of Lithium, Sodium, Potassium, Rubidium, and Caesium. Preferably Alkali metal halide is selected from Lithium, Sodium, Potassium halides. More preferably Alkali metal halide is Lithium bromide.

Alkaline earth metal halide as used in the present invention is selected from halides of Beryllium, Magnesium, Calcium, Strontium, Barium, and Radium. Preferably Alkaline earth metal halide is selected from Magnesium, Calcium halides.

Solvent as defined in the present invention are selected from water or “alcohol solvents” such as methanol, ethanol, n-propanol, isopropanol, n-butanol and t-butanol and the like or “hydrocarbon solvents” such as benzene, toluene, xylene, heptane, hexane and cyclohexane and the like or “ketone solvents” such as acetone, ethyl methyl ketone, diethyl ketone, methyl tert-butyl ketone, isopropyl ketone and the like or “esters solvents” such as methyl acetate, ethyl acetate, propyl acetate, isopropyl acetate, n-butyl acetate, isobutyl acetate, sec-butyl acetate, and the like or “nitrile solvents” such as acetonitrile, propionitrile, butyronitrile and isobutyronitrile and the like or “ether solvents” such as di-tert-butylether, dimethylether, diethylether, diisopropyl ether, 1,4-dioxane, methyltert-butylether, ethyl tert-butyl ether, tetrahydrofuran, 2-methyl tetrahydrofuran, 2-methoxyethanol and dimethoxyethane, or “Amide solvents” such as formamide, DMF, DMAC, N-methyl-2-pyrrolidone, N-methylformamide, 2-pyrrolidone, 1-ethenyl-2-pyrrolidone, haloalkanes such as dichloromethane, 1,2-dichloroethane and chloroform, “Amine solvents” selected from diethylenetriamine, ethylenediamine, morpholine, piperidine, pyridine, quinoline, tributylamine, diisopropyl amine and/or mixtures thereof.

The term “salts” as used herein refers to salts which are known to be non-toxic and are commonly used in the pharmaceutical literature. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenylsubstituted alkanoic acids, hydroxyalkanoic and hydroxyalkandioic acids, aromatic acids, aliphatic and aromatic sulfonic acids, may also be used. Such salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfite, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

The groups Z as defined herein is selected from fluoro, chloro, bromo and iodo, mesyloxy, tosyloxy, trifluoromethylsulfonyloxy, nonafluorobutylsulfonyloxy, (4-bromo-phenyl)sulfonyloxy, (4-nitro-phenyl)sulfonyloxy, (2-nitro-phenyl)sulfonyloxy, (4-isopropyl-phenyl)sulfonyloxy, (2,4,6-tri-isopropyl-phenyl)sulfonyloxy, (2,4,6-trimethyl-phenyl)sulfonyloxy, (4-rertbutyl-phenyl)sulfonyloxy and (4-methoxy-phenyl)sulfonyloxy. Preferably, Z is selected from the group comprising iodo, bromo, chloro, mesyloxy, tosyloxy, (4-nitro-phenyl)sulfonyloxy and (2-nitro-phenyl)sulfonyloxy. More preferably, Z is selected from the group comprising mesyloxy, tosyloxy, trifluoromethylsulfonyloxy and (4-nitro-phenyl)sulfonyloxy.

Alkyl triphenylphosphonium salts as used in the present invention is selected from methyltriphenylphosphonium bromide, methyltriphenylphosphonium iodide ethyltriphenylphosphonium bromide, ethyltriphenylphosphonium iodide, propyltriphenylphosphonium bromide, 2-propynyltriphenyphosphonium bromide, isopropyltriphenylphosphonium iodide, butyltriphenylphosphonium bromide, pentyltriphenylphosphonium bromide, isopentyltriphenylphosphonium bromide etc. Preferably methyltriphenylphosphonium bromide.

Base as used in the present invention is selected from either inorganic base like alkali metal hydroxides such as sodium hydroxide, potassium hydroxide, lithium hydroxide; alkali metal carbonates such as sodium carbonate, potassium carbonate, cesium carbonate and lithium carbonate; Alkali metal bicarbonates such as sodium bicarbonate and potassium bicarbonate; alkali metal alkoxides such as sodium methoxide, potassium methoxide, sodium tertiary butoxide, potassium tertiary butoxide or mixtures thereof or Silicon-based amides, such as sodium and potassium bis(trimethylsilyl)amide, Lithium hexamethyldisilazide, Sodium hexamethyldisilazide and potassium hexamethyldisilazide or organic bases such as LDA (lithium diisopropylamide), triethylamine, triethanolaminetributylamine, N-methylmorpholine, N,N-diisopropylethylamine, di-n-propylamine, N-methylpyrrolidine, pyridine, 4-(N,N-dimethylamino)pyridine, morpholine, imidazole, 2-methylimidazole, 4-methylimidazole, 1,4-diazabicycloundec-7-ene (DBU), 1,5-diazabicyclo[4.3.0]non-5-ene (DBN), 1,4-diazabicyclo[2.2.2]-octane (DABCO) and the like.

In a preferred embodiment, the present invention provides an improved process for the preparation of Efinaconazole of Formula (I)

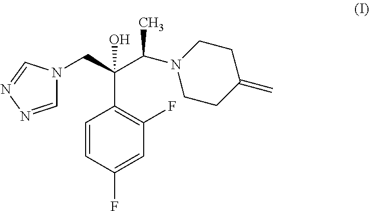 [SMILES: C=C1CCN([C@H](C)[C@](O)(Cn2cnnc2)c2ccc(F)cc2F)CC1]

or its salts, which comprises reacting triazole compound of Formula (III) or its salts

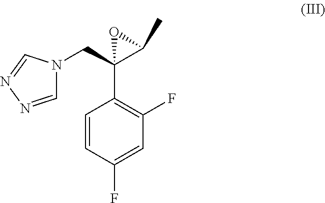 [SMILES: C[C@@H]1O[C@@]1(Cn1cnnc1)c1ccc(F)cc1F]

with HCl salt of compound of Formula (IVA)

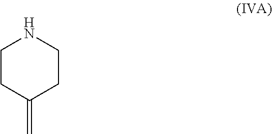

in the presence of alkali metal halide or alkaline earth metal halide in a solvent to give Efinaconazole or its salts.

In another preferred embodiment, the present invention provides novel process for the preparation of Efinaconazole of Formula (I)

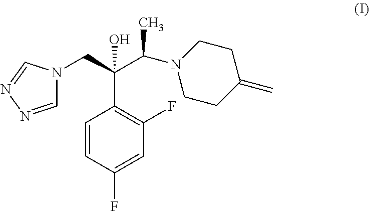 [SMILES: C=C1CCN([C@H](C)[C@](O)(Cn2cnnc2)c2ccc(F)cc2F)CC1]

or its salts, which comprises converting compound of Formula (IIB) or its salts

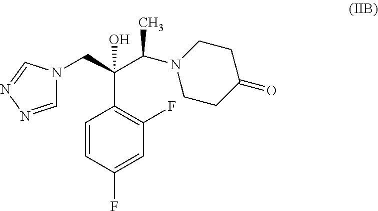 [SMILES: C[C@@H](N1CCC(=O)CC1)[C@](O)(Cn1cnnc1)c1ccc(F)cc1F]

in the presence of alkyl triphenylphosphonium salt, base in a solvent to give Efinaconazole or its salts.

In yet another preferred embodiment, the present invention provides novel process for the preparation of Efinaconazole of Formula (I)

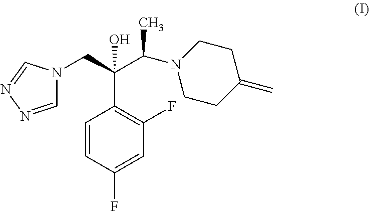 [SMILES: C=C1CCN([C@H](C)[C@](O)(Cn2cnnc2)c2ccc(F)cc2F)CC1]

or its salts, which comprises the following steps:
a) reacting triazole compound of Formula (III) or its salts

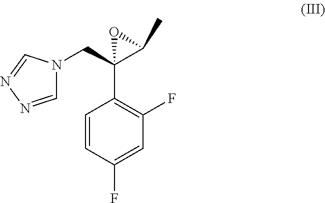 [SMILES: C[C@@H]1O[C@@]1(Cn1cnnc1)c1ccc(F)cc1F]

with HCl salt of compound of Formula (IVC)

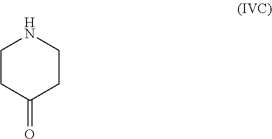

in the presence of a base in a solvent to give compound of Formula (IIB) or its salts; and

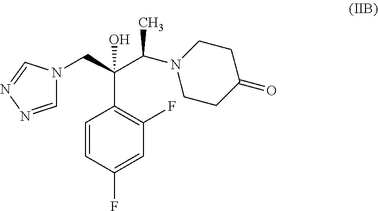 [SMILES: C[C@@H](N1CCC(=O)CC1)[C@](O)(Cn1cnnc1)c1ccc(F)cc1F]

b) converting compound of Formula (IIB) or its salts in the presence of alkyl triphenylphosphonium salt, base in a solvent to give Efinaconazole or its salts.

In yet another preferred embodiment, the present invention provides novel process for the preparation of Efinaconazole of Formula (I)

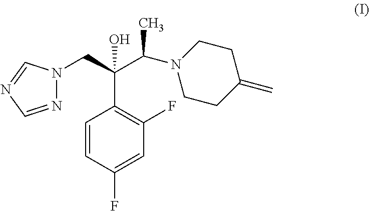 [SMILES: C=C1CCN([C@H](C)[C@](O)(Cn2cncn2)c2ccc(F)cc2F)CC1]

or its salts, which comprises the following steps:
a) reacting compound of Formula (V) or its salts

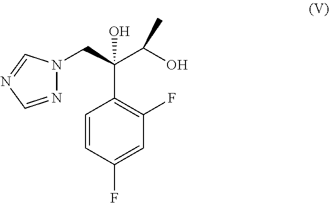 [SMILES: C[C@@H](O)[C@](O)(Cn1cncn1)c1ccc(F)cc1F]

with a suitable reagent in the presence of a base in a solvent to give compound of Formula (VIA) or its salts

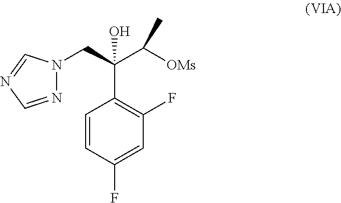 [SMILES: C[C@@H](OS(C)(=O)=O)[C@](O)(Cn1cncn1)c1ccc(F)cc1F]

b) reacting compound of Formula (VIA) or its salts with compound of Formula (IVC) or its salts

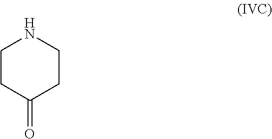

in the presence of a base in a solvent to give compound of Formula (IIB) or its salts; and

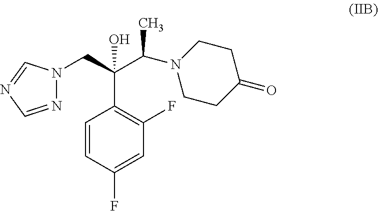 [SMILES: C[C@@H](N1CCC(=O)CC1)[C@](O)(Cn1cncn1)c1ccc(F)cc1F]

c) converting compound of Formula (IIB) or its salts in the presence of alkyl triphenylphosphonium salt, base in a solvent to give Efinaconazole or its salts.

In yet another preferred embodiment, the present invention provides novel process for the preparation of compound of Formula (IIB)

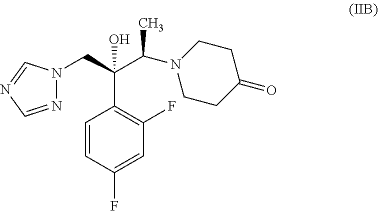 [SMILES: C[C@@H](N1CCC(=O)CC1)[C@](O)(Cn1cncn1)c1ccc(F)cc1F]

or its salts, which comprises reacting triazole compound of Formula (III) or its salts

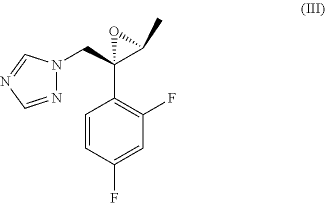 [SMILES: C[C@@H]1O[C@@]1(Cn1cncn1)c1ccc(F)cc1F]

with HCl salt of compound of Formula (IVC)

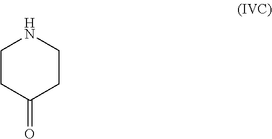

in the presence of a base in a solvent to give compound of Formula (IIB) or its salts.

In yet another embodiment, the present invention provides novel process for the preparation of compound of Formula (IIB)

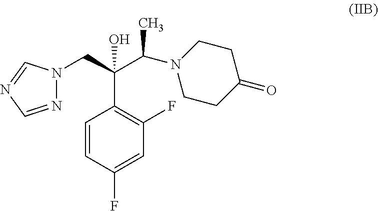 [SMILES: C[C@@H](N1CCC(=O)CC1)[C@](O)(Cn1cncn1)c1ccc(F)cc1F]

or its salts, which comprises the following steps:
a) mesylating compound of Formula (V) or its salts

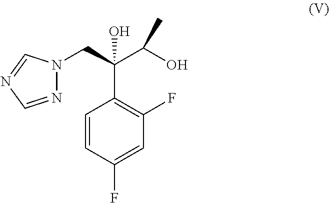 [SMILES: C[C@@H](O)[C@](O)(Cn1cncn1)c1ccc(F)cc1F]

with a mesylating agent in the presence of a base in a solvent to give compound of (VIA) or its salts

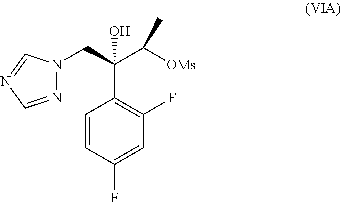 [SMILES: C[C@@H](OS(C)(=O)=O)[C@](O)(Cn1cncn1)c1ccc(F)cc1F]

b) reacting compound of Formula (VIA) or its salts with compound of Formula (IVC) or its salts

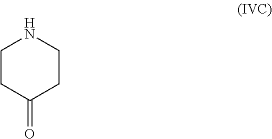

in the presence of a base in a solvent to give compound of Formula (IIB) or its salts.

In yet another preferred embodiment, the present invention provides novel compound of Formula (IIB) or its salts.

In yet another preferred embodiment, the present invention provides highly pure compound of Formula (IIB) in its crystalline or amorphous form.

In yet another embodiment, the present invention provides the use of compound of Formula (IIB) or its salts in the preparation of Efinaconazole.

The present invention is further illustrated by the following examples which are provided merely to be exemplary of the inventions and is not intended to limit the scope of the invention. Certain modifications and equivalents will be apparent to those skilled in the art and are intended to be included within the scope of the present invention.

### Examples

### Example-1: Preparation of Efinaconazole

### Stage-01:

MDC solvent (5 vol) was taken in a clean and dry RBF and added the 4-Methylene piperidine hydrochloride (150 gm) and cooled to 0-5° C., 48% NaOH solution (2 eq) was charged and stirred for 1 hr. The R.M. was quenched with DM water and separated the MDC layer and distilled out completely to get the 4-Methylenepiperidine (100 gm).

### Stage-02:

4-Methylenepiperidine (100 gm), triazole compound of Formula III (100 gm) and 3 Volume of Methyl isobutyl ketone solvent (MIBK) were taken in to a clean and dry R.B.F and added the Lithium Bromide (70 gm) at RT stirred for 30 min and at RT.

The R.M. temp was raised up to 80-85° C. and stirred for 10-12 hrs and the R.M. was cooled to RT and poured in to ice water and extracted with 10 volume of 2-Methyl THF (2-MTHF) solvent and distilled out the solvents under Vacuum and purified with IPA and water to get the crude Efinaconazole compound.

The crude Efinaconazole compound is purified with ethyl acetate and silica gel to get the pure Efinaconazole compound.

### Example-2: Preparation of Efinaconazole

### Stage-01:

MDC solvent (5 vol) was taken in a clean and dry RBF and added the 4-Methylene piperidine hydrochloride (150 gm) and cooled to 0-5° C., 48% NaOH solution (2 eq) was charged and stirred for 1 hr. The R.M. was quenched with DM water and separated the MDC layer and distilled out completely to get the 4-Methylenepiperidine (100 gm).

### Stage-02:

4-Methylenepiperidine (100 gm), triazole compound of Formula III (100 gm) and 3 Volume of acetonitrile solvent (ACN) were taken in to a clean and dry R.B.F and added the Lithium Bromide (70 gm) at RT stirred for 30 min and at RT.

The R.M. temp was raised up to 95-100° C. and stirred for 18-20 hrs and the R.M. was cooled to RT and poured in to IPA+DM water mixture to get the crude Efinaconazole compound.

This crude Efinaconazole compound is purified with ethyl acetate and silica gel to get the pure Efinaconazole compound.

### Example-3: Preparation of Efinaconazole

The triazole compound of Formula III (250 gm) was added to a 10 Volume of N-Methyl morpholine solvent (NMM) in a clean and dry R.B.F, and added the Lithium Bromide (500 gm) at RT stirred for 30 min, and added the 4-methylenepiperidine mono hydrochloride (400 gm) at RT

The R.M. temp was raised up to 90-95° C. and stirred for 36 hrs and the R.M. was cooled to RT and poured in to ice water and extracted with 10 volume of ethyl acetate solvent and distilled out the solvents under Vacuum to get the crude Efinaconazole.

The crude Efinaconazole was added to IPA and water to get the pure Efinaconazole (140 gm).

### Example-4: Preparation of Compound of Formula IIB

Take the Triazole compound of Formula III (100 gm) and acetonitrile (1 lit) solvent in to clean and dry R.B.F and added the Lithium hydroxide (200 gm) at RT stirred for 30 min and added the piperidin-4-one mono hydrochloride of Formula IVC (150 gm) at RT

The R.M. temp raised up to 90-95° C. and stirred for 12 hrs and the R.M. was cooled to RT and poured in to ice water and extracted with ethyl acetate solvent and distilled out the solvents under vacuum to get the crude compound of Formula IIB (120 gm).

### Example-5: Preparation of Efinaconazole

Take the Methyl triphenyl phosphonium bromide (200 gm) and MTBE (1 lit) solvent in to clean and dry R.B.F and added the sodium tert butoxide (200 gm) at RT stirred for 1-2 hrs at 50-55° C. and cooled to RT and added the compound of Formula IIB (100 gm) and stirred for 10-12 hrs art 50-55° C.

The R.M. was cooled to RT and filtered washed with MTBE solvent and distilled out the solvents to get the crude Efinaconazole compound and purified with IPA and water to get the pure Efinaconazole (50 gm)

We claim:

1. An improved process for the preparation of Efinaconazole of Formula (I) 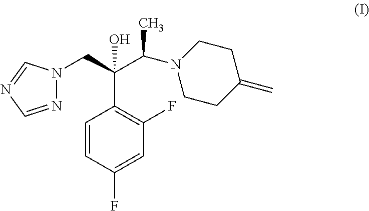 [SMILES: C=C1CCN([C@H](C)[C@](O)(Cn2cncn2)c2ccc(F)cc2F)CC1]
or its salts, which comprises the following steps:
a) i) reacting triazole compound of Formula (III) or its salts 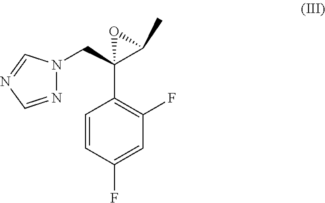 [SMILES: C[C@@H]1O[C@@]1(Cn1cncn1)c1ccc(F)cc1F]
(or)
ii) compound of Formula (VI) or its salts 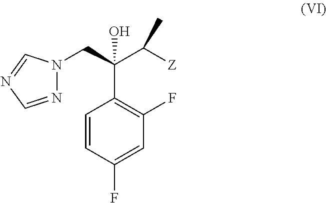
wherein Z is a suitable leaving group, with compound of Formula (IV) or its salts 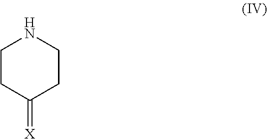
wherein X represents CH₂, O or S, in the presence of an alkali metal halide or alkaline earth metal halides to give compound of Formula (II) or its salts 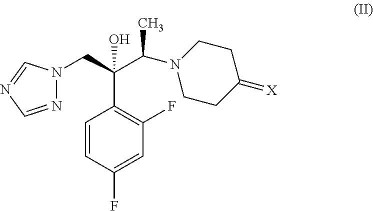
wherein X is as defined above,
b) optionally converting compound of Formula (II) or its salts to Efinaconazole or its salts when X represent O or S.

2. An improved process as claimed in claim 1, wherein the process involves the reaction of triazole compound of Formula (III) or its salts 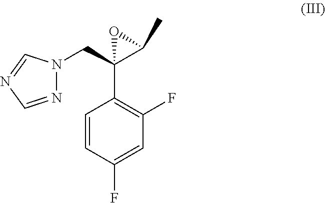 [SMILES: C[C@@H]1O[C@@]1(Cn1cncn1)c1ccc(F)cc1F]
with compound of Formula (IVA) or its salts 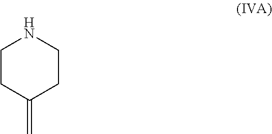
in the presence of an alkali metal halide or alkaline earth metal halides to give Efinaconazole or its salts.

3. An improved process as claimed in claim 1, wherein the process involves the reaction of triazole compound of Formula (VI) or its salts 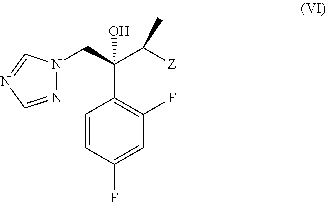
with compound of Formula (IVA) or its salts 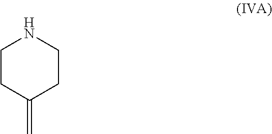
in the presence of an alkali metal halide or alkaline earth metal halides to give Efinaconazole or its salts.

4. An improved process as claimed in claim 1, wherein alkali metal halide is selected from halides of Lithium, Sodium, Potassium, Rubidium, and Caesium and alkaline earth metal halide is selected from halides of Beryllium, Magnesium, Calcium, Strontium, Barium, and Radium.

5. Novel process for the preparation of Efinaconazole of Formula (I) 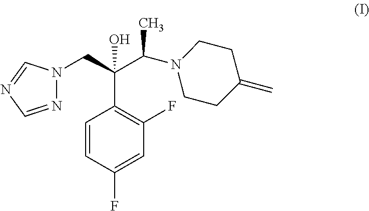 [SMILES: C=C1CCN([C@H](C)[C@](O)(Cn2cncn2)c2ccc(F)cc2F)CC1]
or its salts, which comprises converting compound of Formula (IIA) or its salts 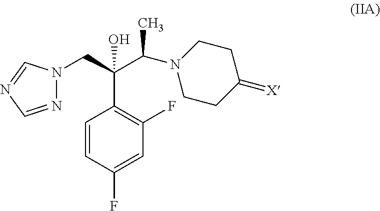
wherein X′ represents O or S, to Efinaconazole or its salts.

6. The process as claimed in claim 5, wherein X′ in compound of Formula (IIA) is preferably O, which is converted to Efinaconazole using alkyl triphenyl phosphonium salt.

7. The process as claimed in claim 6, wherein the alkyl triphenylphosphonium salt is selected from methyltriphenylphosphonium bromide, methyltriphenylphosphonium iodide ethyltriphenylphosphonium bromide, ethyltriphenylphosphonium iodide, propyltriphenylphosphonium bromide, 2-propynyltriphenyphosphonium bromide, isopropyltriphenylphosphonium iodide, butyltriphenylphosphonium bromide, pentyltriphenylphosphonium bromide, isopentyltriphenylphosphonium bromide etc. Preferably methyltriphenylphosphonium bromide.

8. The process as claimed in claim 5, wherein the compound of Formula (IIA) is prepared by reacting compound of Formula (III) 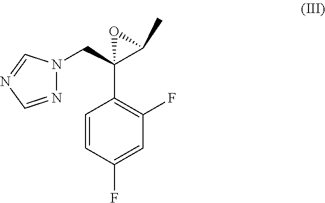 [SMILES: C[C@@H]1O[C@@]1(Cn1cncn1)c1ccc(F)cc1F]
or its salts, with compound of Formula (IVB) 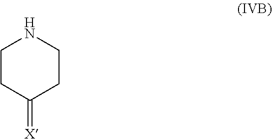
or it salts wherein X′ is as defined above.

9. A process for the preparation of novel compound of Formula (IIA) 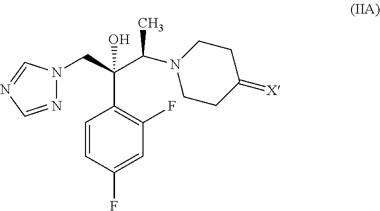
or its salts, wherein X′ represents O or S, which comprises reacting triazole compound of Formula (III) or its salts 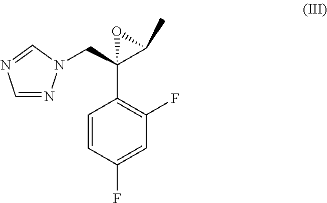 [SMILES: C[C@@H]1O[C@@]1(Cn1cncn1)c1ccc(F)cc1F]
(or) compound of Formula (VI) or its salts 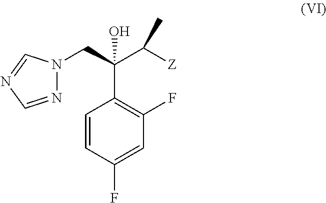
wherein Z is a suitable leaving group, with compound of Formula (IVB) or its salts 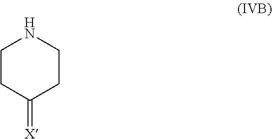
wherein X′ is as defined above, to give compound of Formula (IIA) or its salts.

10. The process as claimed in claim 9, wherein compound of Formula (III) is reacted with compound of Formula (IVB) in the presence of a base in a solvent to give compound of Formula (IIA).

11. The process as claimed in claim 10, wherein the solvent is selected from water or methanol, ethanol, n-propanol, isopropanol, n-butanol and t-butanol or benzene, toluene, xylene, heptane, hexane and cyclohexane or acetone, ethyl methyl ketone, diethyl ketone, methyl tert-butyl ketone, isopropyl ketone or methyl acetate, ethyl acetate, propyl acetate, isopropyl acetate, n-butyl acetate, isobutyl acetate, sec-butyl acetate, or acetonitrile, propionitrile, butyronitrile and isobutyronitrile or di-tert-butylether, dimethylether, diethylether, diisopropyl ether, 1,4-dioxane, methyltert-butylether, ethyl tert-butyl ether, tetrahydrofuran, 2-methyl tetrahydrofuran, 2-methoxyethanol and dimethoxyethane, or formamide, DMF, DMAC, N-methyl-2-pyrrolidone, N-methylformamide, 2-pyrrolidone, 1-ethenyl-2-pyrrolidone, dichloromethane, 1,2-dichloroethane and chloroform, or diethylenetriamine, ethylenediamine, morpholine, piperidine, pyridine, quinoline, tributylamine, diisopropyl amine and/or mixtures thereof.

12. The process as claimed in claim 10, wherein the base is selected from either inorganic base like alkali metal hydroxides such as sodium hydroxide, potassium hydroxide, lithium hydroxide; alkali metal carbonates such as sodium carbonate, potassium carbonate, cesium carbonate and lithium carbonate; Alkali metal bicarbonates such as sodium bicarbonate and potassium bicarbonate; alkali metal alkoxides such as sodium methoxide, potassium methoxide, sodium tertiary butoxide, potassium tertiary butoxide or mixtures thereof or Silicon-based amides, such as sodium and potassium bis(trimethylsilyl)amide, Lithium hexamethyldisilazide, Sodium hexamethyldisilazide and potassium hexamethyldisilazide or organic bases such as LDA (lithium diisopropylamide), triethylamine, triethanolaminetributylamine, N-methylmorpholine, N,N-diisopropylethylamine, di-n-propylamine, N-methylpyrrolidine, pyridine, 4-(N,N-dimethylamino)pyridine, morpholine, imidazole, 2-methylimidazole, 4-methylimidazole, 1,4-diazabicycloundec-7-ene (DBU), 1,5-diazabicyclo[4.3.0]non-5-ene (DBN), 1,4-diazabicyclo[2.2.2]-octane (DABCO) and the like.

13. The process as claimed in claim 9, wherein compound of Formula (VI) is reacted with compound of Formula (IVB) in the presence of a base in a solvent to give compound of Formula (IIA).

14. The process as claimed in claim 13, wherein the solvent is selected from water or methanol, ethanol, n-propanol, isopropanol, n-butanol and t-butanol or benzene, toluene, xylene, heptane, hexane and cyclohexane or acetone, ethyl methyl ketone, diethyl ketone, methyl tert-butyl ketone, isopropyl ketone or methyl acetate, ethyl acetate, propyl acetate, isopropyl acetate, n-butyl acetate, isobutyl acetate, sec-butyl acetate, or acetonitrile, propionitrile, butyronitrile and isobutyronitrile or di-tert-butylether, dimethylether, diethylether, diisopropyl ether, 1,4-dioxane, methyltert-butylether, ethyl tert-butyl ether, tetrahydrofuran, 2-methyl tetrahydrofuran, 2-methoxyethanol and dimethoxyethane, or formamide, DMF, DMAC, N-methyl-2-pyrrolidone, N-methylformamide, 2-pyrrolidone, 1-ethenyl-2-pyrrolidone, dichloromethane, 1,2-dichloroethane and chloroform, or diethylenetriamine, ethylenediamine, morpholine, piperidine, pyridine, quinoline, tributylamine, diisopropyl amine and/or mixtures thereof.

15. The process as claimed in claim 13, wherein the base is selected from either inorganic base like alkali metal hydroxides such as sodium hydroxide, potassium hydroxide, lithium hydroxide; alkali metal carbonates such as sodium carbonate, potassium carbonate, cesium carbonate and lithium carbonate; Alkali metal bicarbonates such as sodium bicarbonate and potassium bicarbonate; alkali metal alkoxides such as sodium methoxide, potassium methoxide, sodium tertiary butoxide, potassium tertiary butoxide or mixtures thereof or Silicon-based amides, such as sodium and potassium bis(trimethylsilyl)amide, Lithium hexamethyldisilazide, Sodium hexamethyldisilazide and potassium hexamethyldisilazide or organic bases such as LDA (lithium diisopropylamide), triethylamine, triethanolaminetributylamine, N-methylmorpholine, N,N-diisopropylethylamine, di-n-propylamine, N-methylpyrrolidine, pyridine, 4-(N,N-dimethylamino)pyridine, morpholine, imidazole, 2-methylimidazole, 4-methylimidazole, 1,4-diazabicycloundec-7-ene (DBU), 1,5-diazabicyclo[4.3.0]non-5-ene (DBN), 1,4-diazabicyclo[2.2.2]-octane (DABCO) and the like.

16. The process as claimed in claim 9, wherein compound of Formula (VI) is prepared by converting compound of Formula (V) or its salts 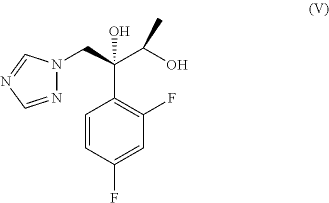 [SMILES: C[C@@H](O)[C@](O)(Cn1cncn1)c1ccc(F)cc1F]
to compound of Formula (VI) or its salts.

17. Novel compound of Formula (IIA) or its salts. 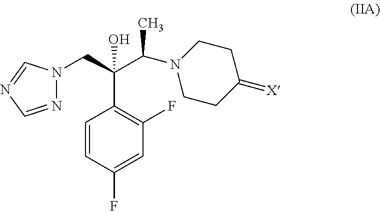
wherein X′ represents O or S.

18. Novel compound of Formula (IIA) or its salts as claimed in claim 17 is further converted to Efinaconazole or its salts.
